(19) United States
(12) Patent Application Publication
(10) Pub. No.: US 2025/0275801 A1
(43) Pub. Date: Sep. 4, 2025

(54) A SURGICAL DEVICE WITH AIRFLOW AND LIGHT

(71) Applicant: Surgis Clearview LP, Saint John (CA)

(72) Inventor: Jorge Albertal, Buenos Aires (AR)

(21) Appl. No.: 18/710,135

(22) PCT Filed: Nov. 29, 2022
(86) PCT No.: PCT/IB2022/000714

§ 371 Date: May 14, 2024

Related U.S. Application Data
(60) Provisional application No. 63/284,209, filed on Nov. 30, 2021.

(51) Int. Cl. A61B 18/00; A61B 90/30

(52) U.S. Cl. CPC A61B 18/00; A61B 90/30; A61B 2018/00178; A61B 2018/00595; A61B 2218/008

(57) ABSTRACT

An electrocautery unit for connecting to a handle of an electrocautery device, the unit comprises a body, a device connector, a conductive connector, a light unit, an airflow unit and an electrode. The body has a proximal end and a distal end. A device connector is located at the proximal end of the body to connect to an elecocautery device. A conductive connector extends from the device connector to the electrode, which is located at the distal end of the body, to provide power the electrode. The airflow unit has at least one airflow intake aperture at the distal end of the body. Also at the distal end of the body is the light source of the light unit.

### CROSS REFERENCE TO RELATED APPLICATION

This application claims the benefit of U.S. Provisional Application No. 63/284,209, filed on Nov. 30, 2022, which is incorporated by reference herein in its entirety.

### FIELD OF INVENTION

The present invention is related generally to a medical apparatus and an accessory for an electrocautery system, and particularly to an electrocautery tool having an airflow unit proximate the distal end of the tool to remove smoke from surgical sites during surgical procedures. The present invention is also related generally to a medical apparatus and an electrocautery system and particularly to an electrocautery tool having a lighting component and an airflow unit proximate the distal end thereof to provide light at, and remove smoke from, surgical sites during surgical procedures, especially deep surgical procedures.

### BACKGROUND OF THE INVENTION

The production of smoke, aerosols, plume, gas, collectively referred to as “smoke” hereafter, in electrosurgical procedures is nearly inevitable. Not only does smoke reduce visibility of the surgical site, it may be toxic or otherwise unpleasant. Surgeons often use a smoke evacuation device to remove the toxic smoke produced, for example, after cutting tissue with an electrode. However, the constraints of certain surgical procedures prevent placement of the air suction nozzle close to the tissue, thus hindering an effective smoke evacuation. This is especially evident in electrosurgical procedures within small surgical keyhole cavity, where the limited incision opening and surgical cavity prohibits any equipment from being inserted or placed closed to the actual surgical site. When smoke occurs, the surgeon usually waits until the smoke is sufficiently removed before proceeding. This pause often occurs multiple times during a single procedure and thus prolonging the procedure.

Accordingly, it is desirable to provide an improved electrocautery apparatus or system that overcomes drawbacks and inadequacies of known methods and systems.

### SUMMARY OF THE INVENTIONS

Generally speaking, in accordance with an embodiment of the invention, a device comprises a body, an airflow unit, a light unit, and an electrode. Thus, once connected to an electrocautery handle, the device may provide electrosurgery, illumination of the surgical site, as well as evacuation of the plume from the surgical site all in one. The device preferably also provides a cooling function. The proximal end of the body preferably connects to a handle of a device via the handle's receiving aperture for an electrode. Therefore, the device may be used with existing standard electrocautery handles. The airflow unit includes an intake aperture proximate the distal end of the body, and more specifically, proximate the electrode tip.

Preferably, the light unit is positioned such that the intake aperture of the airflow unit is proximate the light source, for example, an LED (light-emitting diode). Preferably, the airflow unit preferably removes smoke and heat emitted by the light source. In accordance with an embodiment of the invention, the light source emits a light that is coaxial with the body, and the electrode extends from outside of the central axis of the light and the body inward, such that the electrode tip terminates at the central axis.

Certain embodiments of the invention further include a cooling mechanism to remove heat emitted from the light unit. In accordance with an embodiment, the light unit is located within the airflow unit such that air flows past, and around, the light unit, creating a cooling effect. Another embodiment of the invention provides heatsinks located between the light source and the airflow channel of the airflow unit.

Another embodiment of the invention provides a device having a body, an airflow unit, a light unit, and an electrode-receiving element. Thus, the device may be connected to an electrode. The device may alternatively be connected to forceps or other suitable devices.

An embodiment of the invention provides an electrocautery kit comprising one or more electrodes and an elongated body. The body has a first connecting element at one end constructed and arranged to connect to a handle of a device, preferably at the handle's electrode receiving component. A second connecting element at the opposite end of the body connects to the electrode in the kit. The body further comprises an airflow unit having an intake aperture proximate the electrode when the electrode is connected to the body. The body further comprises a light unit having a light source proximate the intake aperture. The light unit is constructed and arranged to emit a light that preferably is coaxial to the body's central axis lengthwise. When the electrode is connected to the body, the electrode's central axis is not coaxial with the body's central axis. However, the electrode is constructed and arranged to have an electrode tip that extends toward the central axis of the body. Alternatively, the second connecting element may be constructed and arranged to extend the electrode from outside of the central axis toward the central axis.

Still other objects and advantages of the invention will in part be obvious and will in part be apparent from the specification. Other features and advantages of this invention will become apparent in the following detailed description of exemplary embodiments of this invention with reference to the accompanying drawings.

### BRIEF DESCRIPTION OF THE DRAWINGS

For a fuller understanding of the invention, reference is made to the following description taken in connection with the accompanying drawing, in which:

FIG. 1 is a perspective side view of a device in accordance with an embodiment of the invention;

FIG. 2 is a side perspective view of the device of FIG. 1 having a portion of the body wall removed;

FIG. 3 is a partial cross-sectional view of the device of FIG. 1;

FIG. 4 is a rear perspective view of the device of FIG. 1;

FIG. 5 is a side perspective view of a device in accordance with an embodiment of the invention;

FIG. 6 is a side perspective view of the device of FIG. 5 attached to a handle;

FIG. 7 is a rear perspective view of a device in accordance with an embodiment of the invention;

FIG. 8 is a detailed view of a device connector and airflow exit of a device in accordance with an embodiment of the invention;

FIG. 9 is a perspective side view of a device in accordance with an embodiment of the invention;

FIG. 10 is a side perspective view of the device of FIG. 9 having a portion of the body wall removed;

FIG. 11 is a perspective side view of a device in accordance with an embodiment of the invention;

FIG. 12 is a perspective view of the device of FIG. 11 with a variety of electrodes attached thereto;

FIG. 13 is a side perspective view of a device in accordance with an embodiment of the invention having an intake aperture distally farther than a light source;

FIG. 14 is a cross-sectional view of a device in accordance with an embodiment of the invention having an enclosed light unit;

FIG. 15 is an exploded view of the device of FIG. 14;

FIG. 16 is a perspective view of a device in accordance with an embodiment of the invention with an electrode receiving element;

FIG. 17 illustrates how the device of FIG. 16 is attached to a handle

FIG. 18 is a perspective view of the device of FIG. 16 connected to an electrode and a handle;

FIG. 19 is a perspective view of a device in accordance with an embodiment of the invention with an electrode receiving element;

FIG. 20 illustrates how the device of FIG. 19 is attached to a handle

FIG. 21 is a perspective view of the device of FIG. 19 connected to an electrode and a handle;

FIG. 22 is a side perspective view of a device in accordance with an embodiment of the invention;

FIG. 23 is a partial top perspective view of a device in accordance with an embodiment of the invention;

FIG. 24 is a partial top perspective view of a device in accordance with an embodiment of the invention; and

FIG. 25 is a front perspective view of a device in accordance with an embodiment of the invention.

### DETAILED DESCRIPTION OF THE PREFERRED EMBODIMENTS

Certain exemplary embodiments of the present invention will now be described with reference to the drawings. The invention is generally directed to a device having a body, a device connector for removably connecting to an electrocautery handle, preferably a standard electrocautery handle having means to connect to an electrode power source, such that the device retrofits electrocautery handles currently available.

Preferably, the device connector connects to the handle at a connection for electrodes. More specifically, standard electrocautery handles, also referred to as “pencils,” generally have a cavity for receiving the proximal end of an electrode which is generally rod-shaped. The cavity and the proximal end of the electrode both include a conductive element, thus permitting electricity to travel therebetween, namely, from the handle to the electrode when the electrode is inserted into the handle. Likewise, the device connector of the device described herein comprises a conductive material such that electricity can flow from the handle to the electrode attached to the device. Thus, the electrode may be used to cut, dissect, cauterize, or other procedure desired.

The device preferably includes an airflow unit comprising an intake aperture, an airflow channel, and an airflow exit. Preferably, as a procedure is being performed and smoke is being generated, air from the vicinity of the tissue on which the procedure is being performed, enters the airflow unit of the device through its intake aperture, flows through the airflow channel, and exits through the airflow exit.

Typically, during procedures during which electrocautery devices are used, smoke evacuators are provided separately from the electrocautery device. The surgeon may hold the evacuation wand with the non-operating hand, or another person may hold it in place to evacuate the smoke from the surgical site. If the surgeon holds the evacuation wand, it occupies one of his hands. If another person holds the evacuation wand, the surgeon must direct the person when and where to move it. Alternatively, the person must guess and move it without first receiving direction, being careful not to hit the electrode or damaging the tender tissue. Moreover, the tip of the smoke evacuator, such as an evacuation wand, which provides a suctioning force, is often positioned at about 15 cm or more away from the surgical site while the procedure is being performed. Alternatively, the evacuation wand may be brought closer to the tissue, but this usually requires that the surgeon to pause while smoke is being evacuated from the surgical site.

In contrast, intake aperture 121 of device 10 is located closer to electrode tip 161 than 15 cm. Preferably, intake aperture 121 is within 4 cm, more preferably within 2 cm from electrode tip 161. Since electrode tip 161 contacts the tissue, intake aperture 121 will be within 4 cm, and more preferably within 2 cm from the surgical site. Thus, smoke may be more efficiently evacuated than a device positioned 15 cm away, and the amount of smoke escaping from the surgical site may be reduced. The need for an evacuation wand, which may get in the way of the procedure, is preferably eliminated. Additionally, because smoke may be continuously removed closer to the surgical site, it may be unnecessary for the surgeon to pause while the smoke is being evacuated as frequently or at all. Furthermore, because of its proximity to the surgical site, a lower suction force may be sufficient to obtain a similar rate of evacuation as a system in which the evacuation wand is held 15 cm from the surgical site.

Furthermore, exemplary embodiments of the airflow unit of device include an airflow exit through which smoke exits the airflow unit. When there is no force applied, air may flow in either direction between the intake aperture and airflow exit The airflow exit may be connected to a vacuum source, such as a standard or otherwise available evacuation system, to provide a suctioning force to draw the smoke from the surgical site into the intake aperture of the airflow unit and out through the airflow exit. Since the device preferably connects to existing electrocautery handles as well as existing evacuation systems, the device may be used to retrofit existing systems simply by attaching the device to both. Thus, a surgeon may use a handle and evacuation system that the surgeon is familiar with, which may make the transition easier and more cost-effective than a specialized handle or evacuation system.

Additionally, the device preferably also includes a light source proximate, more

preferably adjacent to, the intake aperture and thus in close proximity (within 2 cm) to the surgical site. This arrangement will preferably provide improved lighting at the surgical site compared to overhead lights and headlights without requiring a separate light being aimed at the surgical site. Preferably, the light source is coaxial with the body, sharing a common central axis, and the electrode extends from outside of the central axis into the central axis. More preferably, the electrode tip of the electrode terminates at the central axis. Such an arrangement may reduce the creation of shadows by the electrode. Moreover, by having an airflow unit integrated with the electrode and light as provided in the invention described herein, the device preferably reduces, more preferably eliminates, shadows created by an evacuation wand.

In accordance with preferred embodiments of the invention, a heatsink or other cooling mechanism is provided to dissipate heat from the light source. The amount of heat emitted by LEDs, and more specifically high-performance LEDs, may cause discomfort or perhaps even damage to the patient, surgeon, or someone else who may contact the device. Therefore, it may be preferred to provide a heatsink. The heatsink may be separate or a part of the airflow unit. Alternatively, the airflow unit and the light unit may be located within the same chamber, and thus, air flowing through the airflow unit may cool the light unit without the need a heatsink. Adding a heatsink in this alternative may further dissipate the unwanted heat.

The body of the device preferably houses an electrocautery connector connecting the electrode tip to the device connector, the light unit, and the airflow unit, and is made of a thermally insulating material. Therefore, the device is preferably not hot to the touch, and more preferably is cool to the touch, even when the electrode is powered and furthermore when both the electrode and the light are energized.

In accordance with exemplary embodiments of the invention, the length of the device, measured from the distal end of the electrode tip to the proximal end of the device connector, is between 6 cm and 20 cm, more preferably about 13 cm. The intake aperture and the airflow channel preferably have an internal diameter of between 3 mm and 4 mm. In accordance with an exemplary embodiment, the airflow unit provides at least 45 cfm of suction, preferably at least 65 cfm, when the airflow unit is connection to a vacuum source. The airflow unit also preferably connects to standard suction lines currently available, for example a line having a 6.55 mm (0.25 in) internal diameter.

The airflow unit is preferably passive and does not include a power source. Thus, the evacuation system attached thereto may control the rate of airflow or other factures. For example, the evacuation system may be a liquid removal system and suctions out fluids, such as blood, from the surgical site.

### Light Unit Within Airflow Channel

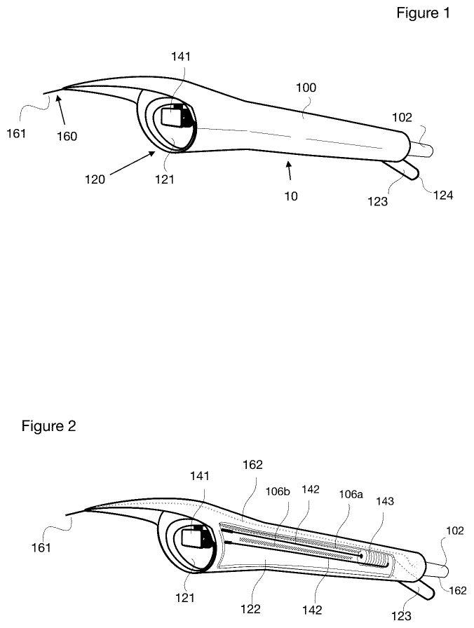

Reference is made to FIGS. 1-4, in which certain embodiments of the invention are shown. Generally, a device 10 includes a body 100 having wall 105 made of a thermally insulating material. Body 100 includes a device connector 102 for removably connecting to handle 110. Preferably, device connector 102 connects to handle 110 at a connection for electrodes and comprises a conductive material such that electricity can flow from handle 110 to electrode 160. As illustrated in FIG. 2, electricity may flow from device connector 102 through electrocautery connector 162 to electrode tip 161. Thus, electrode tip 161 may be used to cut, dissect, cauterize, or other procedure desired.

Device 10 shown also includes an airflow unit 120 comprising an intake aperture 121, an airflow channel 122, and airflow tube 123, and an airflow exit 124. Preferably, smoke enters device 10 through intake aperture 121, flows through airflow channel 122 and subsequently airflow tube 123, and exits through airflow exit 124. Airflow tube 123 may connect to airflow connection tube 130 connected to a vacuum source or another evacuation system which provides the suctioning force. Preferably, airflow tube 123 has a curvature and curves away from device connector 102 so that airflow tube 123 and airflow connection tube 130 extend away from body 100.

The embodiment of device 10 also includes a light unit having a light source 141

proximate intake aperture 121, light cables 142 that extend from batteries 143 along body 100 to light source 141 to provide power from batteries 143 to light source 141. A first insulator 106a preferably prevents the flow of electricity between electrode 160 and light cables 142 and a second insulator 106b preferably prevents the flow of electricity between the two light cables 142. Insulator 106a,b may include any material that does not conduct electricity, such as, but not limited to, plastic. Light holders 144 keep light source 141 in place, maintaining a gap between light source and insulator 106a of body 100, so that air can pass therebetween. Body 100 may further include a structure for securing one or more of the light cables 142 to prevent dislodging or dislocating, for example, from the force of smoke or other content traveling through airflow channel 122.

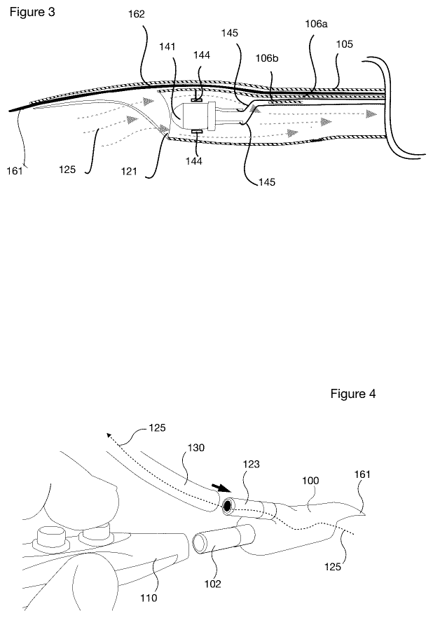

Light sources in general often generate heat, which is potentially dangerous to the patient and may cause tissue damage near the surgical site especially at a close distance as light source 141 of device 10. The embodiment illustrated provides light unit inside of airflow channel 122. Thus, light unit, and particularly light source 141, is preferably cooled as air flows past it. Furthermore, light source 141 is preferably detached from first insulator 106a so that there is a gap therebetween, thus permitting air preferably to flow through the gap to increase the cooling effect of light source 141 than if light source 141 were attached or placed flushed against first insulator 106a. Potential directions of airflow 125 are illustrated in FIG. 3. Additionally, light unit may include a heatsink to further facilitate heat dissipation.

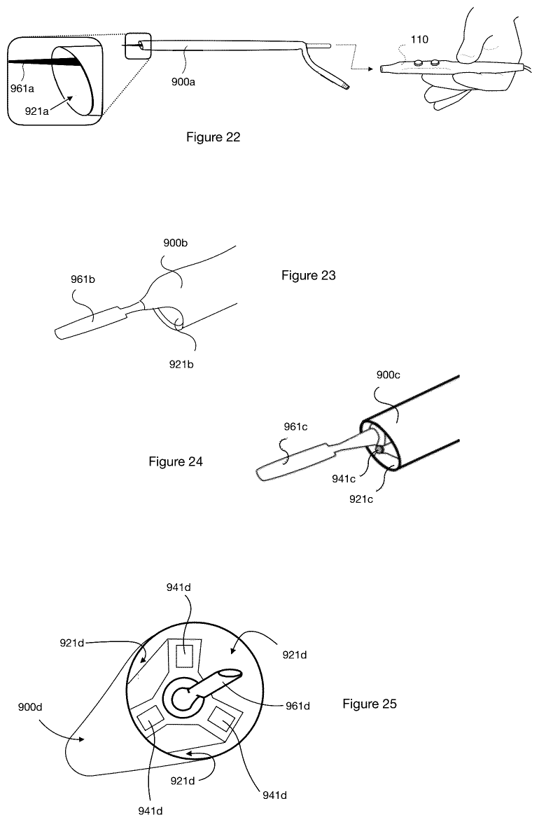

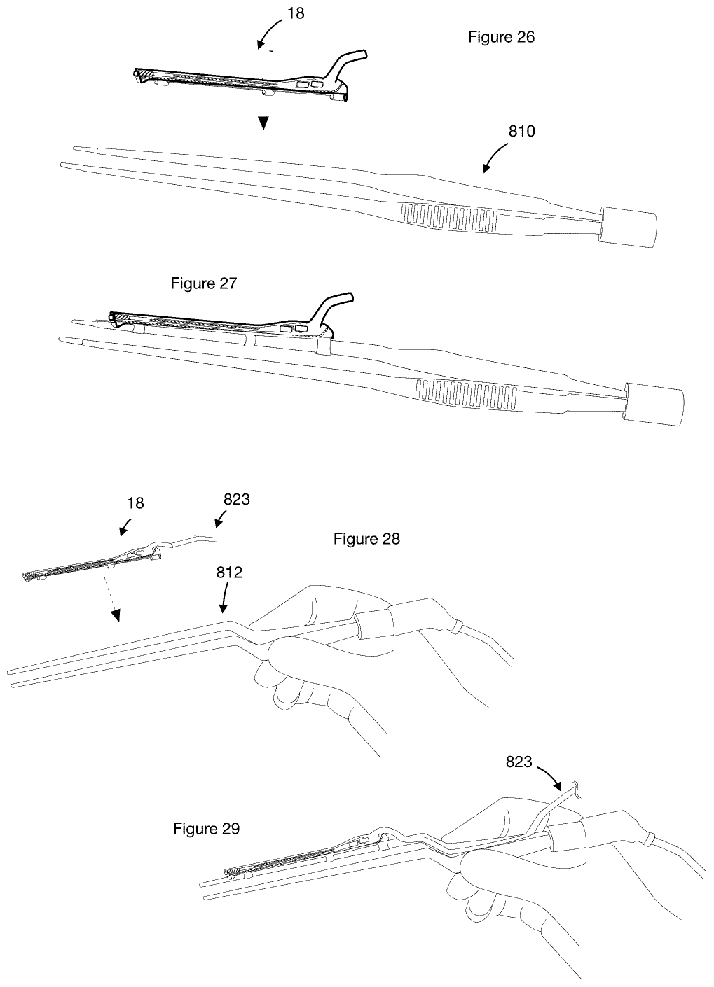

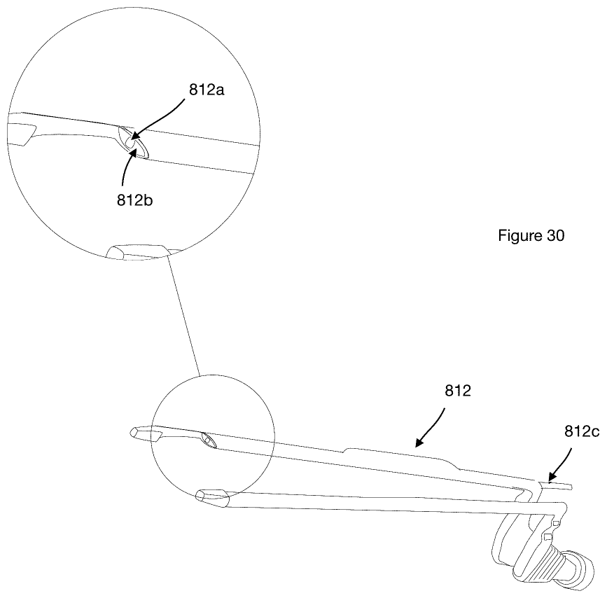

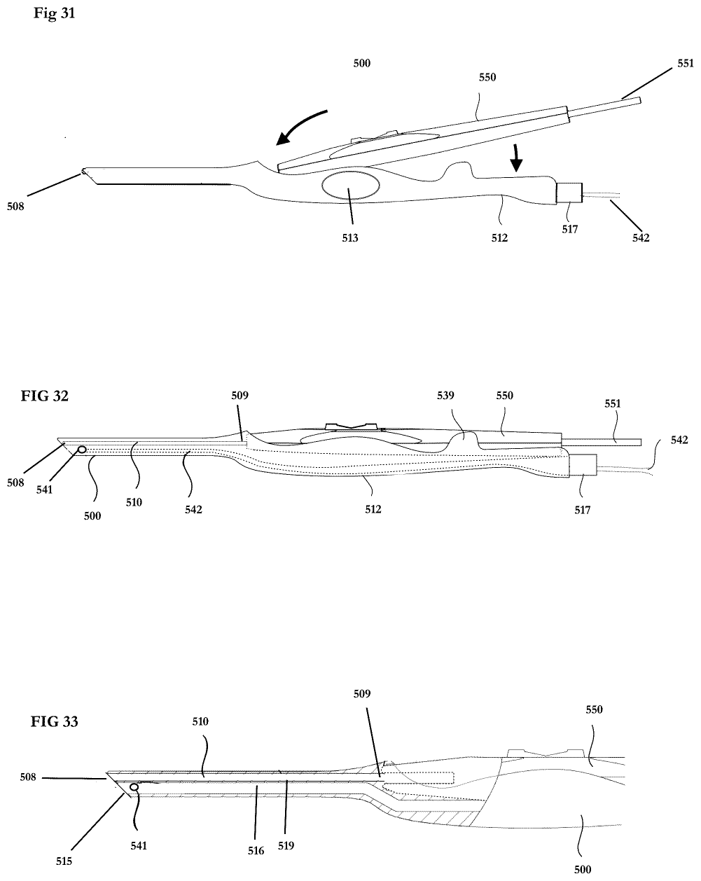

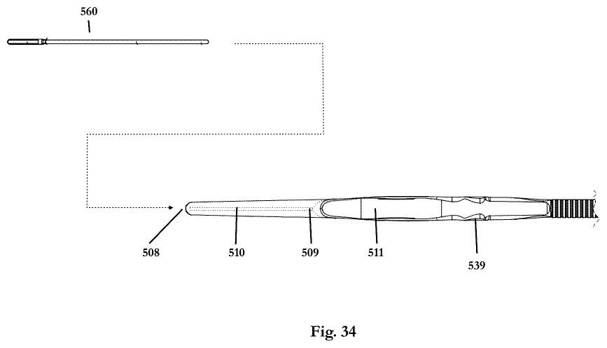

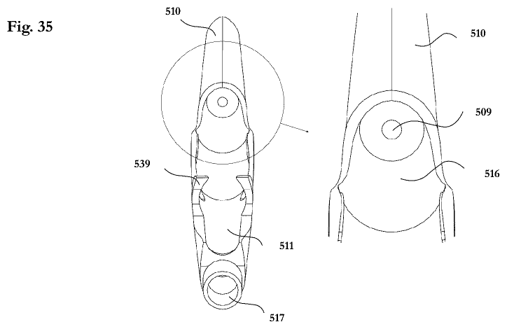

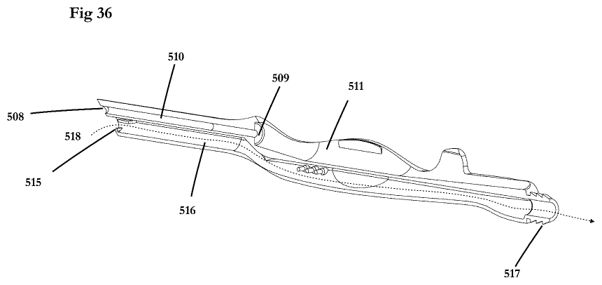

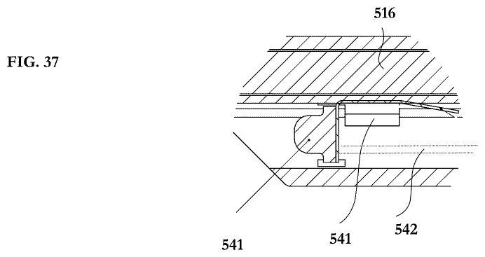

In accordance with another embodiment, both the light unit and the electrode assembly may be positioned and housed within the airflow unit. As illustrated in FIGS. 22, 24 and 25, the electrode tip 961a, c,d may extend from the airflow channel 900a,b,c,d having intake aperture 921a,c,d. As illustrated in FIG. 25, the electrode may be at the center of multiple light sources 941d and intake apertures 921d.

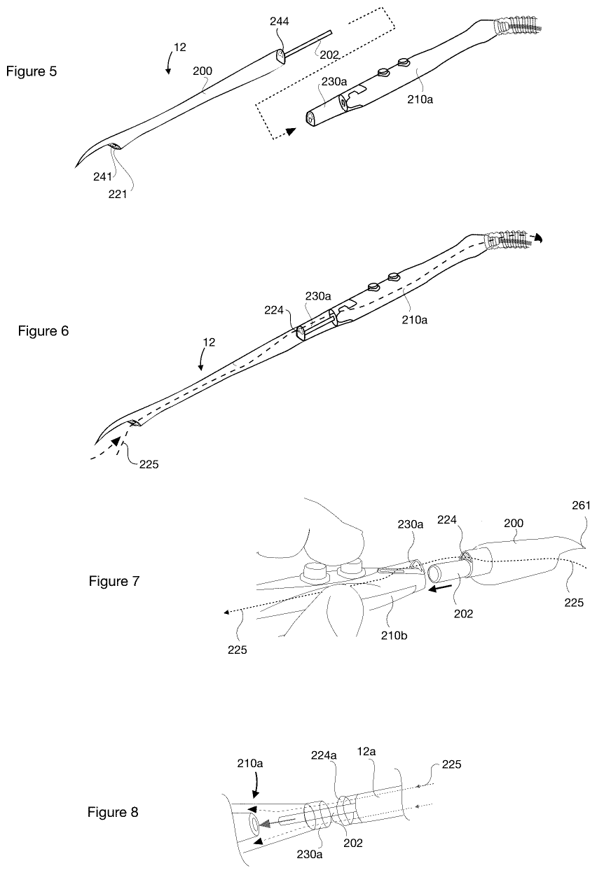

Reference is made to FIGS. 5-7, in which two embodiments of device 12 suitable for use with a smoke evacuation pencil 210a,b is illustrated. As shown, body 200 includes a light source 241 and intake aperture 221, but does not have an airflow tube to connect to an evacuation system. Rather, air flows into intake aperture 221, flows through airflow channel (not shown), exits out of airflow exit 224 and into airflow connector 230a of smoke evacuation pencil 210b as illustrated in FIG. 7. Airflow exit 224 may have a similar shape and arrangement as airflow connector 230b as the embodiment in FIG. 7. It is to be understood that the number of airflow exits 224, its shape, size, location, etc. may be varied as desired to accommodate the corresponding airflow connector of the desired smoke evacuation pencil, and is not limited to that which is illustrated in the drawings.

Alternatively, airflow exit 224 may have a smaller aperture than that of airflow connector 230a as shown in FIG. 6. Thus, the size, position, shape, etc. of airflow exit 124 may be varied. For example, FIG. 8 shows an embodiment of device 12a in which airflow exit 224a is circular and surrounds device connector 202 which connects to airflow connector 230a smoke evacuation pencil 210a. More than one airflow exit may be provided on pencil 210a without deviating from the scope of the invention.

### Light Unit Isolated from Airflow Unit

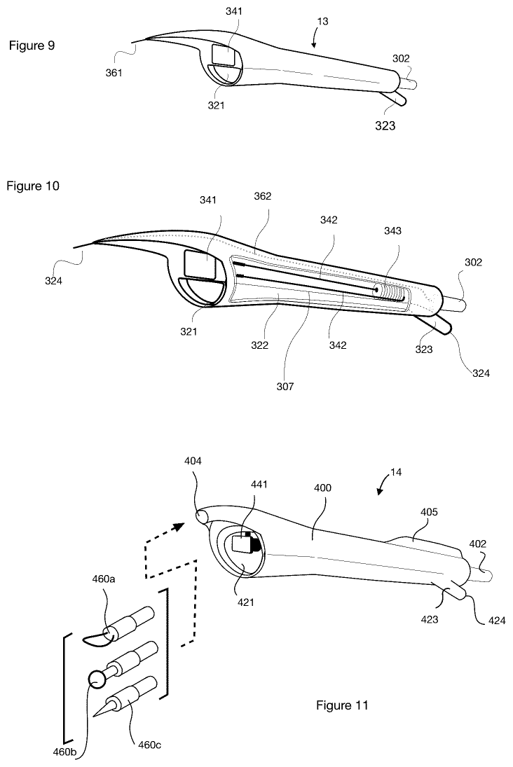

Reference is made to FIGS. 9-10, wherein an embodiment of the invention is illustrated having a light unit separated from the airflow channel by a wall. Device 13 having a body, device connector, airflow unit, light unit, and electrode is shown. Airflow unit has intake aperture 321 leading to airflow channel 322 leading to airflow tube 323 leading to airflow exit 324, thus permitting air to flow in through intake aperture 321 and exit through airflow exit 324. Airflow tube 323 preferably connects to an airflow connection tube or other connection mechanism to a vacuum source or other suction device. Light unit includes light source 341 proximate intake aperture 321 and connected by light cables 342 to batteries 343 for powering light source 341. Device 13 includes an electrode having an electrode tip 361 extending out of body and electrically connected by electrocautery connector 362 to device connector 302. The arrangement of electrode in device 13 may be the same as in device 10, wherein the electrode extends from the exterior of the central of light source 341, which preferably coincides with the central axis of the body of device 13, and the distal end of electrode tip 361 terminates at the central axis of light source 341. Alternatively, as illustrated in FIGS. 22-25, the electrode tip 961a,b,c, d may extend from the body without extending into the central axis of the light source 941a,b,c,d.

As illustrated, device 13 includes divider 307, which creates two separate chambers, one housing the airflow unit and the other housing the light unit. By providing airflow channel 322 separated from the light unit, there are preferably no obstacles that may hinder or slow down the flow of air passing through. Divider 307 preferably is or includes a heatsink to absorb and/or disperse excess heat from light source 341. The heatsink may be a small portion of divider 307, for example, comprising less than half the area of divider 307. Alternatively, it may comprise half or more of the surface area, for example, the entire divider 307 may be a heatsink. Preferably, as air flows through airflow channel 322, it cools the heatsink and facilitates cooling of light source 341. In accordance with another embodiment, divider 307 may comprise a thermally insulating material to prevent the transfer of heat, and the light unit may include a heatsink separate from divider 307. In yet in another alternative, divider 307 may have perforations to allow the passage of air between the two chambers.

Furthermore, whereas the embodiment shown in FIGS. 9-10 include airflow tube 323 having airflow exit 324 at the end thereof, it is to be understood other variations are contemplated without deviating from the scope of the invention, For example, device 13 may include the embodiments of the airflow exit and mechanisms for connecting to a smoke evacuation pencil 210a,b as shown in FIGS. 5-8 instead of or in combination with airflow tube 323.

### Additional Embodiments of the Electrode

Reference is made to FIGS. 11-12, in which additional embodiments of the electrode are illustrated by way of non-limiting example. There are various electrodes that are used in electrosurgery. Any electrode suitable to attach to an electrocautery handle may be compatible with device 14. In the embodiment shown, device 14 comprises body 400 having device connector 402 and electrode connector 404. Generally, electrode connector 404 comprises a cavity constructed and arranged to receive the proximal end 463a,b,c of respective electrode 460a,b,c. Electrode connector 404 preferably has a conductive material and transfers electricity to electrode 460a,b,c when proximal end 463a, b,c is inserted therein.

The electrodes described herein may be a variety of electrodes and are not limited to the examples illustrated herein. For example, a variety shapes and sizes of electrodes may be used according to the desired surgical procedure. For example, the electrode may be a blade for cutting, and the specific size and shape of the blade may differ depending on the location of the surgery and the type of tissue to be cut. The electrode may be curved, angular, comprise a loop, a ball, a hook, or any other shape desired, preferably with the electrode tip extending toward the central axis of the light source such that the electrode tip is located along the central axis.

In the embodiments illustrated, the light unit includes batteries to independently power the light source. The batteries may be positioned in the body in such a way that they do not bulge radially outward of the body, as shown in FIG. 2. Alternatively, as shown in FIG. 11, the body may have a battery housing 405 protruding radially outward in which batteries for the light unit is located. Battery housing 405 may include an external access so that a spent battery may be replaced by a new battery. However, alternate embodiments in which the light source is powered by the handle or by an exterior power source are contemplated without deviating from the scope of the invention.

### Enclosed Light Unit

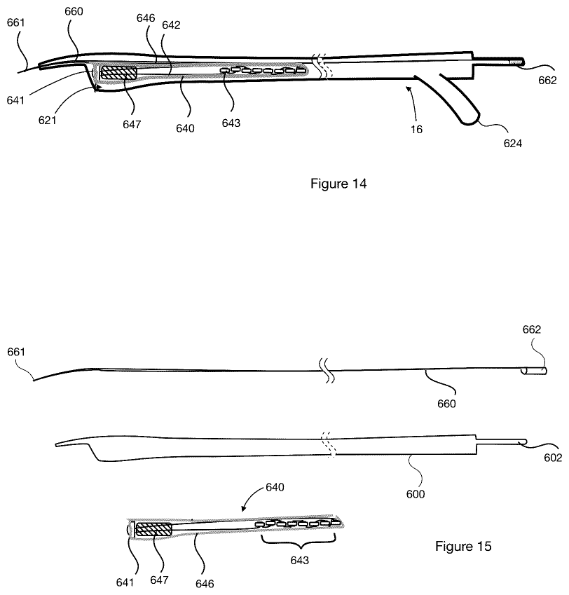

Reference is made to FIGS. 14-15, which illustrate an embodiment of the invention having an enclosed light unit 640. As shown, device 16 has a body 600 housing light unit 640, electrode 660, and intake aperture 621 and airflow exit 624 for the flow of smoke or other substance through body 600. Light unit 640 includes light source 641 connected to batteries 643 via light cables 642 located within light unit housing 646. The embodiment also includes a heatsink 647 within light unit housing 646 proximate light source 641. As shown in FIG. 15, Device 16 has a device connector 602 which electrically connects a handle to the electrocautery connector 662 to transmit electricity from the handle to electrode tip 661. An enclosed light unit within the airflow unit may be desirable when non-gas matters, such as blood, is being evacuated through the airflow channel. Body 600 may include may include an external access to remove, replace or or exchange light unit 640.

### Device Without Conductive Connector

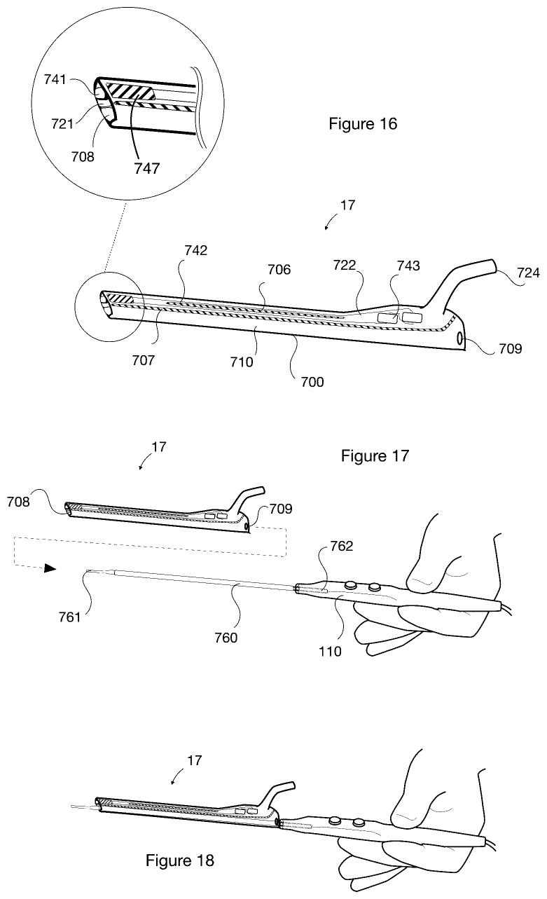

FIGS. 16-18 illustrate an embodiment of the invention in which device 17 does not include an electrode within the body 700 of the device, but rather, includes a channel for receiving electrode 760, preferably a standard electrode currently available in the market. Body 700 has an electrode tip aperture 708, an electrocautery connector aperture 709, and an electrode channel 710 therebetween. Thus, electrode 760 is preferably inserted into body 700 by inserting electrode tip 761 into electrocautery connector aperture 709, through electrode channel 710 and out electrode tip aperture 708. When properly positioned, electrocautery connector 762 protrudes through electrocautery connect aperture 709. Preferably, electrode 760 is connected to handle 110 by inserting electrocautery connector 762 into the receiving cavity of handle 110, after which device 17 is attached by inserting electrode 760 into body 700 as described above. Alternatively, electrode 760 may be inserted into body 700 prior to being connected to handle 110.

Therefore, existing handles and electrodes may be retrofit with device 17 to provide light and suction proximate electrode tip 761. As shown, device 17 further includes a light source 741 connected by light cables 742 to batteries 743, and a heatsink 747 which dissipates heat from light source 741. Body 700 also provides an intake aperture 721 through which smoke or other substances may be drawn into an airflow channel before existing out of body 700 through airflow exit 724. A divider 707 separates electrode channel 710 from airflow channel 722. Preferably, divider 707 is an insulator in addition to insulator 706 positioned between two light cables.

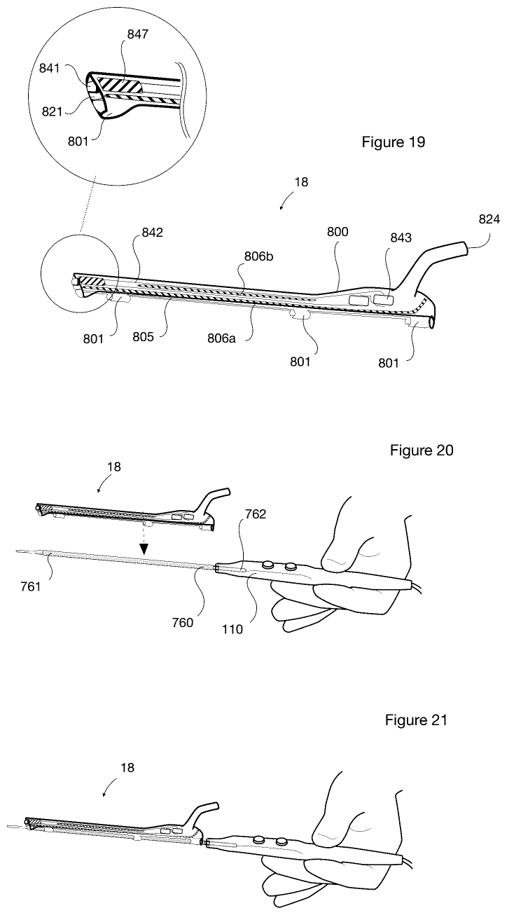

FIGS. 19-21 illustrate an embodiment in which device 18 includes clips 801 for attaching to electrode 760. As shown in FIG. 20, device 18 may be placed adjacent to electrode 760 and connect device 18 onto electrode 760 such that clips 801 hold device 18 and electrode 760 together. As shown in FIG. 19, device 18 further includes a light source 841 connected by light cables 842 to batteries 843, and a heatsink 847 which dissipates heat from light source 841. Body 800 also provides an intake aperture 821 through which smoke or other substances may be drawn into an airflow channel before existing out of body 800 through airflow exit 824. Body 800 further includes insulators 806a,b. As shown in greater detail in FIG. 19, insulator 806a is located within the wall of body 800. Preferably, device 18 includes a plurality of clips 801, more preferably at least three: one each on the proximal and distal ends of body 800, and at least one in between. More preferably, device 18 has four clips 801 as shown or more which may improve stability.

The embodiments illustrated in FIGS. 16-21 may alternatively be used with other devices with or without an electrocautery handle. For example, device 17,18 may be attached to forceps, for example electrosurgical forceps. Device 17 may receive a leg of the forceps in electrode channel 710. Device 18 may clip onto a leg of the forceps. Preferably, when device 17,18, is connected to the forceps, light source 741,841 and intake aperture 721,821 are proximate the tip of the forceps, thus providing light to and removing smoke from the surgical site.

An exemplary use of device 18 on two different types of forceps 810, 812 are shown in FIGS. 26-29. As shown in FIGS. 28-29, airflow connection tube 823 may bend to accompany the shape of forceps 812. Airflow connection tube 823 may have a shape corresponding to the shape of forceps 812 as shown or airflow connection tube 823 may be flexible and thus adaptable to the shape of the instrument on which device 18 is attached. Alternatively, the airflow tube of device 18 itself may be arranged to bend along the forceps leg, preferably further including one or more clips or another connection mechanism to maintain the airflow tube in position on the forceps leg on which the device 18 is attached.

Whereas FIGS. 26-29 illustrate an exemplary use with electrocautery forceps, it is to be understood that embodiments of the invention may be utilized with other types of forceps including forceps that are not constructed to be energized, as well as other suitable devices. Furthermore, whereas the illustrated embodiments show airflow connection tube 823 that bends according to the shape of forceps 812, the device 17, 18 itself may have one or more bends or curves to fit the device with which it is used or for other reasons desired.

An alternate embodiment illustrated in FIG. 30 includes a light and airflow unit integrated within a leg of forceps 812. Proximate the distal tip of forceps 812 is a light unit 812a, such as an LED. Light unit 812 is located at or proximate intake aperture 812b, preferably arranged such that air can surround most of, if not completely, the light unit 812 as it enters intake aperture 812b and flows within the airflow channel. Airflow exit 812c is shown proximate the distal end of the forceps leg having the light and airflow unit.

Whereas the embodiments illustrated are directed toward a combined light and airflow device, it is contemplated to provide a light unit on one leg and an airflow unit on the other leg of the forceps, both proximate the distal end of the forceps. The light unit and the airflow unit may be built into the forceps or alternatively, attached as with devices 17, 18 to retrofit existing forceps. The attachable units may be separate and attach one unit individually per leg, or they may be connected to form a unitary device. The light unit preferably includes on or more heatsinks as described above.

Whereas the examples of light source illustrated herein is an LED, it is to be understood that various sources of light may be used, depending on the desired size, color, temperature, amount of heat it emits, current, etc.

The handle preferably includes a switch element for activating the electrode. The switch element may control the type of electrical current supplied to the electrode tip. Switch element may comprise buttons, which control the current running through the handle and supplied to the electrode tip, to effect electrocoagulation and/or electrocauterization. Whereas one current selection may allow the electrode tip to cut; another current selection may allow the electrode tip to perform coagulation. By selecting between the buttons, the surgeon may change the tool function from cutting to coagulation easily. A switch for activating the light unit may be provided on the body or externally to the device.

Additionally, whereas in certain embodiments described above, the electrode's electrocautery connector is housed within the device, such that the device connector is inserted into and comes into electrical contact with the handle, the device connector may have an aperture through which the electrocautery connector extrudes and contacts the handle directly.

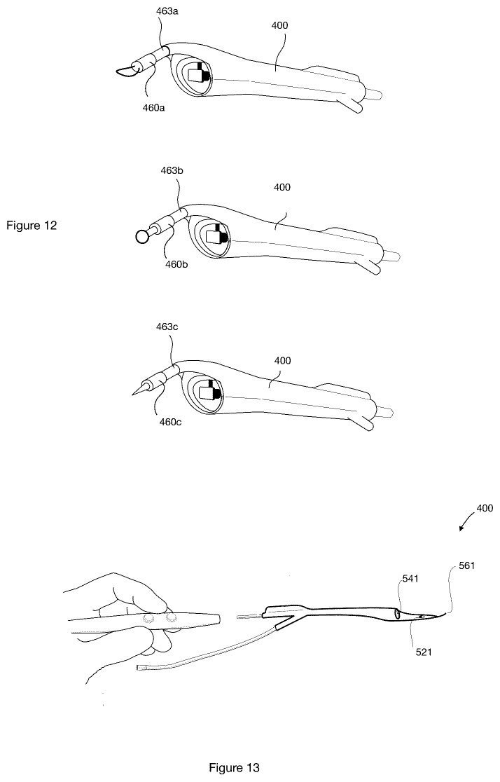

The examples provided are merely exemplary, as a matter of application specific to design choice, and should not be construed to limit the scope of the invention in any way. Thus, while there have been shown and described and pointed out novel features of the present invention as applied to preferred embodiments thereof, it will be understood that various omissions and substitutions and changes in the form and details of the disclosed invention may be made by those skilled in the art without departing from the spirit of the invention. For example, as illustrated in FIG. 13, the intake aperture 521 may be located farther distally on the body of the device, closer to the electrode tip 561, than light source 541.

Additionally, other alterations can be made. As a way of non-limiting example, the shape and size of the body, electrode, intake aperture, airflow channel, airflow tube, airflow exit, light source, device connector may be varied without deviating from the scope of the invention. Whereas the embodiments are described herein for removing smoke from a surgical site, it may be used to remove fluids or any other element that can fit through the airflow unit. Moreover, whereas the embodiments include a light source at the distal end of the device, it is to be understood that the same embodiments without a light source are contemplated as well and do not deviate from the scope of the invention. Any combination of the alternatives and modifications mentioned above as well as those not specified herein may be made without deviating from the scope of the invention as a matter of application specific to design choice. It is the intention, therefore, to be limited only as indicated by the scope of the claims appended hereto.

It is also to be understood that the following claims are intended to cover all of the generic and specific features of the invention herein described and all statements of the scope of the invention, which, as a matter of language, might be said to fall therebetween.

1. An electrocautery device comprising:
a body having a proximal end and a distal end;
a device connector disposed at the proximal end of the body configured to connect to a handle of an electrocautery pencil;
an airflow unit having at least one airflow channel extending between the proximal end and the distal end of the body, said airflow unit further comprising an airflow intake aperture proximate the distal end of the body and an airflow exit aperture at the proximal end of the body;
a light unit having a light source proximate the distal end of the body;
an electrode disposed at the distal end of the body, said electrode is configured and positioned further away from the proximal end of the body than the airflow intake aperture and the light source; and
a conductive connector constructed and configured to transmit electricity from the device connector to the electrode when the device is connected to an electrocautery pencil.

2. The device of claim 1, wherein electrode is configured and positioned to extend from the airflow unit.

3. The device of claim 1 wherein the light unit further comprising a power source and a light cable connecting the power source to the light source.

4. The device of claim 1 further comprising at least one heatsink.

5. The device of claim 1 further comprising at least one insulator.

6. The device of claim 1 wherein the light unit is configured and positioned within the airflow channel.

7. The device of claim 1 wherein the light unit further comprising a light holder configured to hold the light source as to allow air to flow around the light source.

8. The device of claim 1 further comprising an external access for removing the light unit.

9. The device of claim 1 further comprising a divider disposed lengthwise within the body for creating a first compartment as the airflow channel and a second compartment for housing the lighting unit.

10. An elongated electrocautery device having a proximal end and a distal end comprising:
a handle receptacle for receiving an electrocautery handle;
an electrode channel for receiving and positioning an electrocautery electrode for connection to the electrocautery handle received by the handle receptacle;
and an airflow channel to allow air to pass from the distal end of the elongated electrocautery device to the proximal end of the elongated electrocautery device;
wherein said distal end of the elongated electrocautery device having a first aperture defining one end of said electrode channel, and a second aperture defining one end of said airflow channel proximate said first aperture.

11. The device of claim 10 further comprising a light source positioned in said airflow channel proximate said second aperture to allow the light source to emit light out of the second aperture.

12. The device of claim 10 further comprising an airflow exit connector defining the proximal end of said airflow channel.

13. The device of claim 10 further comprising at least one heatsink.

14. The device of claim 10 further comprising at least one insulator.

15. The device of claim 10, further comprising a holding portion proximate said handle receptacle to hold the received electrocautery handle securely.

16. An airflow electrocautery device comprising:
a hollow body having a proximal end, and a distal end;
an airflow channel defined by an air intake aperture disposed at the distal end of the hollow body and an air exit aperture disposed at the proximal end of the hollow body;
a light unit positioned within the airflow channel, said light unit having a light source proximate the distal end of the body;
an arm extending distally beyond the distal end of the hollow body, having an electrode connector at the distal end of the arm for receiving a variety of electrode tips.
a device connector disposed at the proximal end of the body configured to connect to a handle of an electrocautery pencil; and
a conductive connector extending from the device connector to the electrode connector.

17. The device of claim 16 further comprising a second air intake aperture positioned on the arm proximate the electrode connector.

18. The device of claim 16 further comprising an insulator.

19. The device of claim 16 further comprising a heatsink.

20. The device of claim 16 further comprising light holder configured to hold the light source as to allow air to flow around the light source.
